(19) United States
(12) Patent Application Publication
(10) Pub. No.: US 2009/0023762 A1
(43) Pub. Date: Jan. 22, 2009

(54) Substituted 2-Aminopyrimidine-4-Ones, Their Pharmaceutical Compositions And Their Use In The Treatment And/Or Prevention Of Ab-Related Pathologies

(75) Inventors: Stefan Berg, Sodertalje (SE); Jeremy Burrows, Sodertalje (SE); Sven Hellberg, Sodertalje (SE); Katharina Hogdin, Sodertalje (SE); Karin Kolmodin, Sodertalje (SE)

Correspondence Address: PEPPER HAMILTON LLP, 400 BERWYN PARK, 899 CASSATT ROAD, BERWYN, PA, 19312-1183, US

(73) Assignees: ASTRAZENECA AB, Sodertalje (SE); ASTEX THERAPEUTICS LIMITED, Cambridge (GB)

(21) Appl. No.: 12/097,316

(22) PCT Filed: Dec. 18, 2006
(86) PCT No.: PCT/SE06/01434

§ 371 Date: Sep. 5, 2008

Related U.S. Application Data
(60) Provisional application No. 60/752,166, filed on Dec. 19, 2005.

(51) Int. Cl. A61K 31/513; C07D 239/47; A61P 25/28

(52) U.S. Cl. 514/272; 544/319

(57) ABSTRACT

This invention relates to novel compounds having the structural formula (I) below: and to their pharmaceutically acceptable salt, compositions and methods of use. These novel en compounds provide a treatment or prophylaxis of cognitive impairment, Alzheimer Disease, neurodegeneration and dementia.

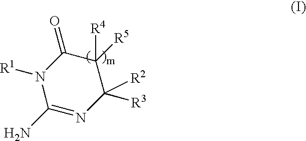

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). O-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO 05/097767, US2005/0282826, WO 06/065277).

The compounds of the present invention show improved properties compared to the potential inhibitors known in the art, e.g. improved hERG selectivity.

### DISCLOSURE OF THE INVENTION

Provided herein are novel compounds of structural formula I:

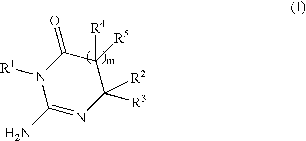

wherein
R¹ is selected from hydrogen, C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl, wherein the C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one, two or three A;
R² is selected from hydrogen, nitro, cyano, -Q-C₁₋₆alkyl, -Q-C₂₋₆alkenyl, -Q-C₂₋₆alkynyl, Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alklheterocyclyl, wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylarl, -Q-C₁₋₆alkylheteroaryl, -Q-heterocyclyl, or -Q-C₁₋₆alkylheterocyclyl is optionally substituted by one, two or three R⁷;
-Q- is a direct bond, —CONH—, —CO—, —CON(C₁₋₆alkyl)-, —CON(C₃₋₆cycloalkyl)-, —SO—, —SO₂—, —SO₂NH—, —SO₂N(C₁₋₆alkyl)-, —SO₂N(C₃₋₆cycloalkyl)-, —NHSO₂—, —N(C₁₋₆alkyl)SO₂—, —NHCO—, —N(C₁₋₆alkyl)CO—, —N(C₃₋₆cycloalkyl)CO— or —N(C₃₋₆cycloalkyl)SO₂—;
R³ is (C(R²⁷)(R²⁸))ₙR⁶, C₂₋₄alkenylR⁶, C₂₋₄alkynylR⁶, C₅₋₇cycloalkenylR⁶, nitro or cyano and if n>1 then each C(R²⁷)(R²⁸) is independent of the others;
R²⁷ and R²⁸ are independently selected from hydrogen, C₁₋₆alkyl, cyano, halo or nitro; or R²⁷ and R²⁸ together form oxo, C₃₋₆cycloalkyl or heterocyclyl;
R⁴ and R⁵ are selected from hydrogen, nitro, cyano, -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl, wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl is optionally substituted by one, two or three R⁷; or
R⁴ and R⁵ may optionally join together to form a C₃₋₇cycloalkyl, C₅₋₇cycloalkenyl or heterocycle ring optionally substituted by one, two or three R⁷; or
R⁴ or R⁵, which are connected to the carbon directly adjacent to the carbon to which R² and R³ are connected, join together with either R² or R³ to form a C₃₋₇cycloalkyl, C₅₋₇cycloalkenyl or heterocycle ring optionally substituted by one, two or three R⁷;
R⁶ is selected from methyl, C₃₋₆cycloalkyl, heterocyclyl, aryl or heteroaryl wherein each of the said methyl, C₃₋₆cycloalkyl, heterocyclyl, aryl or heteroaryl is optionally substituted with between one and four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and four A with the proviso that the bicyclic ring is not an indane, benzo[1,3]dioxole or 2,3-dihydrobenzo[1,4]-dioxine ring system;
R⁷ is selected from halogen, nitro, CHO, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸ (CO)R⁹, O(CO)NR⁸R⁹, NR⁸(CO)OR⁹, NR⁸(CO)NR⁸R⁹, O(CO)OR⁸, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₁₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl, wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and four A with the proviso that said bicyclic ring system is not an indane, benzo[1,3]dioxole or 2,3-dihydrobenzo[1,4]-dioxine ring system;
R¹⁴ is selected from halogen, nitro, CHO, C₀₋₆alkylCN, OC₁₋₆alkylCN, C₀₋₆alkylOR⁸, OC₁₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸(CO)R⁹, O(CO)NR⁸R⁹, NR⁸(CO)OR⁹, NR⁸(CO)NR⁸R⁹, O(CO)OR⁸, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₂₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, OR⁸, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl may be optionally substituted by between one and four A;
R⁸ and R⁹ are independently selected from hydrogen, C₁₋₆alkyl, C₂₋₆alkenyl, C₂₋₆alkynyl, fluoromethyl, difluoromethyl, trifluoromethyl, fluoromethoxy, difluoromethoxy, trifluoromethoxy, C₀₋₆alkylC₃₋₆cycloalkyl, C₀₋₆alkylaryl, C₀₋₆alkylheteroaryl, C₀₋₆alkylheterocyclyl and C₁₋₆alkylNR¹⁰R¹¹, wherein the C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl or C₀₋₆alkylheterocyclyl are optionally substituted by A; or
R⁸ and R⁹ may together form a 4 to 6 membered heterocyclic ring containing one or more heteroatoms selected from N, O or S that is optionally substituted by A; whenever two R⁸ groups occur in the structure then they may optionally together form a 5 or 6 membered heterocyclic ring containing one or more heteroatoms selected from N, O or S, that is optionally substituted by A;
R¹⁰ and R¹¹ are independently selected from hydrogen, C₁₋₆alkyl, C₃₋₆alkenyl, C₃₋₆alkynyl, C₀₋₆alkylC₃₋₆cycloalkyl, C₀₋₆alkylaryl, C₀₋₆alkylheterocyclyl and C₀₋₆alkylheteroaryl, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, C₀₋₆alkylheterocyclyl are optionally substituted by A; or
R¹⁰ and R¹¹ may together form a 4 to 6 membered heterocyclic ring containing one or more heteroatoms selected from N, O or S optionally substituted by A;
m is 1 or 2
n is 0, 1, 2 or 3
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, OC₂₋₆alkylNR¹²R¹³, NR¹²R¹³, CONR¹²R¹³, NR¹²(CO)R¹³, O(CO)C₁₋₆alkyl, (CO)OC₁₋₆alkyl, COR¹², (SO₂)NR¹²R¹³, NSO₂R¹², SO₂R¹², SOR¹², (CO)C₁₋₆alkylNR¹²R¹³, (SO₂)C₁₋₆alkylNR¹²R¹³, OSO₂R¹², SO₃R¹² wherein the C₁₋₆alkyl, C₂₋₆alkenyl, C₂₋₆alkynyl, C₀₋₆alkylaryl, C₀₋₆alkylheteroaryl, C₀₋₆alkylheterocyclyl and C₀₋₆alkylC₃₋₆cycloalkyl groups may be optionally substituted with halo, OSO₂R¹², SO₃R¹², nitro, cyano, OR¹², C₁₋₆alkyl, fluoromethyl, difluoromethyl, trifluoromethyl, fluoromethoxy, difluoromethoxy and trifluoromethoxy;
R¹² and R¹³ are independently selected from hydrogen, C₁₋₆alkyl, C₃₋₆cycloalkyl, aryl, heteroaryl or heterocyclyl wherein said C₁₋₆alkyl, C₃₋₆cycloalkyl, aryl, heteroaryl or heterocyclyl is optionally substituted by one, two or three hydroxy, cyano, halo or C₁₋₃alkyloxy; or
R¹² and R¹³ may together form a 4 to 6 membered heterocyclic ring containing one or more heteroatoms selected from N, O or S optionally substituted by hydroxy, C₁₋₃alkyloxy, cyano or halo;
provided that either any of the aryl or heteroaryl groups in R¹, R², R³, R⁴ or R⁵ is substituted with a OSO₂R⁹, SO₃R⁸, OSO₂R¹² or SO₃R¹² group;
or
provided that when any of the individual aryl or heteroaryl groups in R¹, R², R³, R⁴ or R⁵ are fused with a 4, 5, 6 or 7 membered cycloalkyl, cycloalkenyl or heterocyclyl group to form a bicyclic ring system where the bicyclic ring system is optionally substituted with between one and four A, the bicyclic ring is not an indane, benzo[1,3]dioxole or 2,3-dihydrobenzo[1,4]-dioxine ring system;
or
provided that when R¹ is C₃₋₆alkynyl or C₅₋₇cycloalkenyl, said groups are optionally substituted with one, two or three A;
or
provided that Q is selected from —NHSO₂—, —N(C₁₋₆allyl)SO₂—, —SO₂NH—, —SO₂N(C₁₋₆alkyl)- or —SO₂N(C₃₋₆cycloalkyl)- or —N(C₃₋₆cycloalkyl)SO₂—;
or
provided that R³ is selected from C₂₋₄alkenylR⁶, C₂₋₄alkynylR⁶, C₅₋₇cycloalkenylR⁶ or nitro;
or
provided that R² is selected from nitro, C₂₋₆alkynyl, C₅₋₇cycloalkenyl or C₂₋₆alkenyl group where the C₂₋₆alkynyl, C₅₋₇cycloalkenyl or C₂₋₆alkenyl group is optionally substituted by one, two or three R⁷;
or
provided that R⁴ or R⁵ are independently selected from nitro, C₂₋₆alkynyl, C₅₋₇cycloalkenyl or C₂₋₆alkenyl group where the C₂₋₆alkynyl, C₅₋₇cycloalkenyl or C₂₋₆alkenyl group is optionally substituted by one, two or three R⁷;
or
provided that when Q is —SO— or —SO₂— that the said —SO— or —SO₂— group connect to carbons;
as a free base or a pharmaceutically acceptable salt, solvate or solvate of a salt thereof.

It is to be understood that when m is 1, Formula I represents a 6-membered ring structure and when m is 2, Formula I represents a 7-membered ring structure.

The present invention further provides compositions comprising a compound of formula I, and at least one pharmaceutically acceptable carrier, diluent or excipient.

The present invention further provides methods of modulating activity of BACE comprising contacting the BACE with a compound of formula I.

The present invention further provides methods of treating or preventing an Aβ-related pathology in a patient, comprising administering to the patient a therapeutically effective amount of a compound of formula I.

The present invention further provides a compound described herein for use as a medicament.

The present invention further provides a compound described herein for the manufacture of a medicament.

In a further aspect of the invention, there is provided a compound of formula I, wherein R¹ is C₁₋₆alkyl.

In one embodiment of this aspect, C₁₋₆alkyl is methyl.

In another aspect of the invention, there is provided a compound of formula I, wherein -Q- in R¹ represents a direct bond.

In yet another aspect of the invention, there is provided a compound of formula I, wherein R² is C₁₋₆alkyl.

In one embodiment of this aspect, C₁₋₆alkyl is methyl.

In yet another aspect of the invention, there is provided a compound of formula I, wherein R³ is (C(R²⁷)(R²⁸))ₙR⁶.

In one embodiment of this aspect, n is 0.

In yet another aspect of the invention, there is provided a compound of formula I, wherein R⁶ (of R³) is aryl, substituted with one R⁷.

In yet another aspect of the invention, there is provided a compound of formula I, wherein R⁷ is selected from C₀₋₆alkylaryl, wherein C₀₋₆alkylaryl, is substituted by one or more R¹⁴, or wherein any of the individual aryl groups is fused with a 6 membered heterocyclyl group to form a bicyclic ring system.

In one embodiment of this aspect, said C₀₋₆alkylaryl is phenyl.

In another embodiment of this aspect, R¹⁴ is independently selected from OSO₂R⁸ and OR⁸.

In yet another embodiment of this aspect, R⁸ is C₁₋₆alkyl.

In yet another embodiment of this aspect, said phenyl is fused with a 6 membered heterocyclyl group to form a bicyclic ring system.

In yet another aspect of the invention, there is provided a compound of formula I, wherein R⁴ is hydrogen.

In yet another aspect of the invention, there is provided a compound of formula I, wherein m is 1.

In yet another aspect of the invention, there is provided a compound of formula I, wherein R¹ is C₁₋₆alkyl, -Q- in R² represents a direct bond and R² is C₁₋₆alkyl, R³ is (C(R²⁷)(R²⁸))ₙR⁶, n is 0, R⁶ (of R³) is aryl, substituted with one R⁷, R⁷ is phenyl substituted by one or more R¹⁴, R¹⁴ is independently selected from OSO₂R⁸ and OR⁸, R⁸ is C₁₋₆alkyl, R⁴ is hydrogen and m is 1.

In yet another aspect of the invention, there is provided a compound of formula I, wherein R¹ is C₁₋₆alkyl, -Q- in R² represents a direct bond and R² is C₁₋₆alkyl, R³ is (C(R²⁷)(R²⁸))ₙR⁶, n is 0, R⁶ (of R³) is aryl, substituted with one R⁷, R⁷ is phenyl fused with a 6 membered heterocyclyl group to form a bicyclic ring system, R⁴ is hydrogen and m is 1.

Another embodiment of the present invention provides compounds of formula I comprising the following:
3′-(2-Amino-1,4-dimethyl-6-oxo-1,4,5,6-tetrahydropyrimidin-4-yl)-5-methoxybiphenyl-3-yl methanesulfonate;
2-Amino-6-[3-(3,4-dihydro-2H-chromen-8-yl)phenyl]-3,6-dimethyl-5,6-dihydropyrimidin-4(3H)-one hydrochloride;
   as a free base or a pharmaceutically acceptable salt, solvate or solvate of a salt thereof.

Some compounds of formula I may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

Compounds of the invention can be used as medicaments. In some embodiments, the present invention provides compounds of formula I, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In a further embodiment, the compounds of the present invention are represented by formula (I) or a pharmaceutically acceptable salt thereof as recited herein for use as a medicament.

In a further embodiment, the compounds of the present invention are represented by formula (I) or a pharmaceutically acceptable salt thereof as recited herein in the manufacture of a medicament for the treatment or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia.

In a further embodiment, the compounds of the present invention are represented by a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined herein.

In a further embodiment, the compounds of the present invention are represented by a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined herein.

In a further embodiment, the compounds of the present invention are represented by a method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer Disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined herein and a cognitive and/or memory enhancing agent.

In a further embodiment, the compounds of the present invention are represented by a method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined herein and a choline esterase inhibitor or anti-inflammatory agent.

In a further embodiment, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In a further embodiment, the present invention provides that the mammal or human being treated with a compound of the invention has been diagnosed with a particular disease or disorder, such as those described herein. In these cases, the mammal or human being treated is in need of such treatment. Diagnosis, however, need not be previously performed.

Schizophrenia and other Psychotic Disorder(s) includes but is not limited to Psychotic Disorder(s), Schizophreniform Disorder(s), Schizoaeffective Disorder(s), Delusional Disorder(s), Brief Psychotic Disorder(s), Shared Psychotic Disorder(s), and Psychotic Disorder(s) Due to a General Medical Condition. 2) Dementia and other Cognitive Disorder(s). 3) Anxiety Disorder(s) including but not limited to Panic Disorder(s) Without Agoraphobia, Panic Disorder(s) With Agoraphobia, Agoraphobia Without History of Panic Disorder(s), Specific Phobia, Social Phobia, Obsessive-Compulsive Disorder(s), Stress related Disorder(s), Posttraumatic Stress Disorder(s), Acute Stress Disorder(s), Generalized Anxiety Disorder(s) and Generalized Anxiety Disorder(s) Due to a General Medical Condition. 4) Mood Disorder(s) including but not limited to a) Depressive Disorder(s), including but not limited to Major Depressive Disorder(s) and Dysthymic Disorder(s) and b) Bipolar Depression and/or Bipolar mania including but not limited to Bipolar I, including but not limited to those with manic, depressive or mixed episodes, and Bipolar II, c) Cyclothymiac's Disorder(s), d) Mood Disorder(s) Due to a General Medical Condition. 5) Sleep Disorder(s). 6) Disorder(s) Usually First Diagnosed in Infancy, Childhood, or Adolescence including but not limited to Mental Retardation, Downs Syndrome, Learning Disorder(s), Motor Skills Disorder(s), Communication Disorders(s), Pervasive Developmental Disorder(s), Attention-Deficit and Disruptive Behavior Disorder(s), Feeding and Eating Disorder(s) of Infancy or Early Childhood, Tic Disorder(s), and Elimination Disorder(s). 7) Substance-Related Disorder(s) including but not limited to Substance Dependence, Substance Abuse, Substance Intoxication, Substance Withdrawal, Alcohol-Related Disorder(s), Amphetamines (or Amphetamine-Like)-Related Disorder(s), Caffeine-Related Disorder(s), Cannabis-Related Disorder(s), Cocaine-Related Disorder(s), Hallucinogen-Related Disorder(s), Inhalant-Related Disorder(s), Nicotine-Related Disorder(s)s, Opiod-Related Disorder(s)s, Phencyclidine (or Phencyclidine-Like)-Related Disorder(s), and Sedative-, Hypnotic- or Anxiolytic-Related Disorder(s). 8) Attention-Deficit and Disruptive Behavior Disorder(s). 9) Eating Disorder(s). 10) Personality Disorder(s) including but not limited to Obsessive-Compulsive Personality Disorder(s). 11) Impulse-Control Disorder(s).

Neurodegenerative Disorder(s) includes, but is not limited to, Alzheimer's Disease, Mild Cognitive Impairment, Dementia, Age-Associated Memory Impairment, Age-Related Cognitive Decline, Disorder(s) associated with neurofibrillar tangle pathologies, Dementia due to Alzheimer's Disease, Dementia due to Schizophrenia, Dementia due to Parkinson's Disease, Dementia due to Creutzfeld-Jacob Disease, Dementia due to Huntington's Disease, Dementia due to Pick's Disease, Stroke, Head Trauma, Spinal Injury, Multiple Sclerosis, Migraine, Pain, Systemic Pain, Localized Pain, Nociceptive Pain, Neuropathic Pain, Urinary Incontinence, Sexual Dysfunction, Premature Ejaculation, Motor Disorder(s), Endocrine Disorder(s), Gastrointestinal Disorder(s), and Vasospasm.

Many of the above conditions and disorder(s) are defined for example in the American Psychiatric Association Diagnostic and Statistical Manual of Mental Disorders, Fourth Edition, Text Revision, Washington, D.C., American Psychiatric Association, 2000.

The present invention also includes pharmaceutical compositions that contain, as the active ingredient, one or more of the compounds of the invention herein together with at least one pharmaceutically acceptable carrier, diluent or excipent.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₅₋₆aryl, OC₅₋₆aryl, C(═O)C₁₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl”, “alkylenyl” or “alkylene” used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups. As used herein, “C₁₋₃ alkyl”, whether a terminal substituent or an alkylene (or alkylenyl) group linking two substituents, is understood to specifically include both branched and straight chain methyl, ethyl, and propyl.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used alone or as a suffix or prefix is intended to include both branched and straight-chain alkyne containing aliphatic hydrocarbon groups having from 2 to 12 carbon atoms or if a specified number of carbon atoms is provided then that specific number would be intended. For example “C₂₋₆alkynyl” denotes alkynyl having 2, 3, 4, 5 or 6 carbon atoms. Examples of alkynyl include, but are not limited to, ethynyl, 1-propynyl, 2-propynyl, 3-butynyl, -pentynyl, hexynyl and 1-methylpent-2-ynyl.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 4 to 12 carbons atoms.

As used herein, “cycloalkynlyl” refers to ring-containing hydrocarbyl groups having at least one carbon-carbon triple bond in the ring, and having from 7 to 12 carbons atoms.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form is the N-oxide or S-oxide(s) or a ring nitrogen is optionally quarternized; wherein a ring —NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl, chroman.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used.

As used herein, “in vivo hydrolysable precursors” means an in vivo hydrolysable (or cleavable) ester of a compound of formula Ia or formula Ib that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically labeled compounds of the invention. An “5 isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to 2H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In some embodiments, the present invention provides a compound of formula Ia or formula Ib or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanol amine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group that may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph

Derivatives that are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., -Me, -Et, -nPr, -iPr, -nBu, -sBu, -iBu, -tBu); C₁₋₇-aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound that, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 ng/kg to 10 mg/kg per day. For instance, dosages can be readily ascertained by those skilled in the art from this disclosure and the knowledge in the art.

Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have a beneficial effect in treatment of Alzheimer's Disease and other neurodegenerative diseases associated with elevated levels and/or deposition of Aβ peptide. Therefore it is believed that the compounds of the present invention may be used for the treatment of Alzheimer disease and disease associated with dementia Hence compounds of the present invention and their salts are expected to be active against age-related diseases such as Alzheimer, as well as other Aβ related pathologies such as Downs syndrome and β-amyloid angiopathy. It is expected that the compounds of the present invention would most likely be used in combination with a broad range of cognition deficit enhancement agents but could also be used as a single agent.

### Methods of Preparation

The present invention also relates to processes for preparing the compound of formula I as a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in “Protective Groups in Organic Synthesis”, T. W. Greene, P. G. M Wutz, Wiley-Interscience, New York, 1999. It is understood that microwaves can be used for the heating of reaction mixtures.

### Preparation of Intermediates

The process, wherein R¹, R², R³, R⁴ and R⁵, unless otherwise specified, are as defined hereinbefore, comprises,

(i) Intermediates II, III, IV, V, VI and VII were prepared according to Scheme 1 and the fall experimental details are given in Examples 1-6.

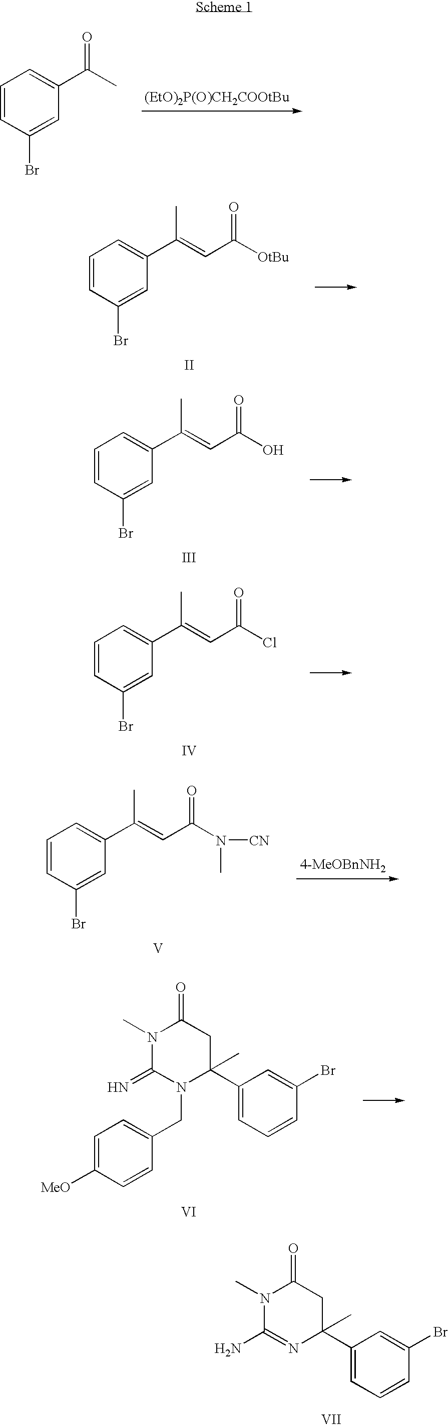

(ii) conversion of a compound of formula VIII to obtain a compound of formula IX, wherein R²⁹ is a group taken from R⁷ or a protected form thereof, Halo represents chloro, bromo or iodo and R³⁰ is a group taken from either R⁸ or R¹²

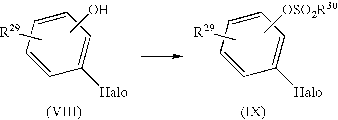

may be carried out by reaction with a suitable reagent such as an alkyl sulfonyl chloride e.g. methane sulfonyl chloride, an alkyl sulfonic anhydride, e.g. trifluoromethanesulfonic anhydride, or a sulfonamide e.g. N-phenyl-bis(trifluoromethanesulfonimide, in the presence of a suitable base such as an organic amine base such as pyridine, 2,6-lutidine, s-collidine, triethylamine, diisopropyl ethylamine, morpholine, N-methylmorpholine, diazabicyclo[5.4.0]undec-7-ene or tetramethylguanidine, or an alkali metal or an alkaline earth metal carbonate such as sodium carbonate, potassium carbonate or calcium carbonate, or potassium phosphate, in a suitable solvent such as dichloromethane, tetrahydrofuran, chloroform, toluene, dimethyl formamide or pyridine at a temperature of −78° C. to 120° C. 4-Dimethylaminopyridine may aid the reaction.

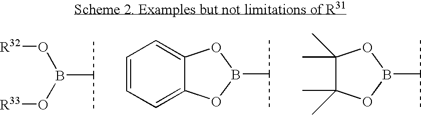

(iii) borylation of a compound of formula X to obtain a compound of formula XI, wherein Halo represents halogen such as iodine, bromine or chlorine, R³¹ may be a group outlined in Scheme 2, wherein R³² and R³³ are groups such as hydrogen, C₁₋₆alkyl, C₂₋₃alkyl, aryl or cycloalkyl and two C₂₋₃alkyl may be fused together to form a 5 or 6 membered boron containing heterocycle and wherein the alkyl, cycloalkyl or aryl moieties may be optionally substituted; R³⁴ includes hydrogen or those definitions covered by R⁷ hereinbefore, provided that the substitutent is compatible with the cross-coupling chemistry. Alternative optionally substituted aromatic and heteroaromatic ring systems in addition to phenyl are also covered within this process.

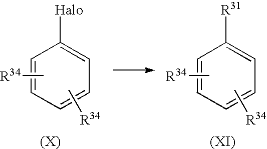

may be carried out by a reaction with:
a) an alkyllithium such as butyllithium, or magnesium, and a suitable boron compound such as trimethyl borate or triisopropyl borate. The reaction may be performed in a suitable solvent such as tetrahydrofuran, hexane or dichloromethane in a temperature range between −78° C. and +20° C.;
or,
b) a suitable boron species such as biscatecholatodiboron, bispinacolatodiboron or pinacolborane in the presence of a suitable palladium catalyst such as palladium(0) tetrakistriphenylphosphine, palladium diphenylphosphineferrocene dichloride or palladium acetate, with or without a suitable ligand such as 2-(dicyclohexylphosphino)biphenyl, and a suitable base, such as a tertiary amine, such as trietylamine or diisopropylethylamine, or potassium acetate may be used. The reaction may be performed in a solvent such as dioxane, toluene, acetonitrile, water, ethanol or 1,2-dimethoxyethane, or mixtures thereof, at temperatures between +20° C. and +160° C.

### Methods of Preparation of End Products

Another object of the invention is a process for the preparation of compounds of general Formula I, wherein R¹, R², R³, R⁴ and R⁵ unless otherwise specified, are defined as hereinbefore, and salts thereof. When it is desired to obtain the acid salt, the free base may be treated with an acid such as a hydrogen halide such as hydrogen chloride, sulphuric acid, a sulphonic acid such as methane sulphonic acid or a carboxylic acid such as acetic or citric acid in a suitable solvent such as tetrahydrofuran, diethyl ether, methanol, ethanol, chloroform or dichloromethane or mixtures thereof, the reaction may occur between −30° C. to +50° C.

These processes comprise;

(a) conversion of a compound of formula XII (for example compound VII above, when Halo represents bromine) to obtain a compound of formula I′ (where I′ is covered within the general definitions of a compound of formula I), wherein Halo represents halogen such as chlorine, bromine or iodine, ring B is defined such that once Halo has been substituted by R⁷ then the resultant final product I′ is covered by a compound of formula I,

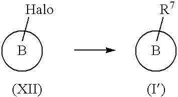

The reaction of process (a) may be carried out by a de-halogen coupling with a suitable compound such as a group of formula XI.

The reaction may be carried out by coupling of a compound of formula XII with an appropriate aryl boronic acid or a boronic ester of formula XI. The reaction may be carried out using a suitable palladium catalyst such as tetrakis(triphenylphosphine)palladium(0), palladium diphenylphosphineferrocene dichloride or palladium(II) acetate, together with, or without, a suitable ligand such as tri-tert-butylphosphine or 2-(dicyclohexylphosphino)biphenyl, or using a nickel catalyst such as nickel on charcoal or 1,2-bis(diphenylphosphino)ethanenickel dichloride together with zinc and sodium triphenylphosphinetrimetasulfonate. A suitable base such as cesium fluoride, an alkyl amine such as triethyl amine, or an alkali metal or alkaline earth metal carbonate or hydroxide such as potassium carbonate, sodium carbonate, cesium carbonate, or sodium hydroxide may be used in the reaction, which may be performed in a temperature range between +20° C. and +160° C., in a suitable solvent such as toluene, tetrahydrofuran, dioxane, dimethoxyethane, water, ethanol or N,N-dimethylformamide, or mixtures thereof.

(b) conversion of a compound of formula XIII to obtain a compound of formula I″ se kommentar ovan (where I″ is covered within the general definitions of a compound of formula I), wherein ring C is defined such that when the —OH is replaced by —OSO₂R⁸ or —OSO₂R¹² then the resultant final product I″ is covered by a compound of formula I,

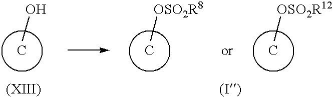

The reaction of process (b) may be carried out by reaction with a suitable reagent such as an alkyl sulfonyl chloride e.g. methane sulfonyl chloride, an alkyl sulfonic anhydride e.g. trifluoromethanesulfonic anhydride, or a sulfonimide e.g. NT-phenyl-bis(trifluoromethanesulfonimide, in the presence of a suitable base such as an organic amine base such as pyridine, 2,6-lutidine, s-collidine, 4-dimethylaminopyridine, triethylamine, diisopropyl ethylamine, morpholine, N-methylmorpholine, diazabicyclo[5.4.0]undec-7-ene or tetramethylguanidine, or an alkali metal or an alkaline earth metal carbonate or hydroxide such as sodium hydroxide, sodium carbonate, potassium carbonate or calcium carbonate, or potassium phosphate, in a suitable solvent such as dichloromethane, tetrahydrofuran, chloroform, toluene, dimethyl formamide or pyridine at a temperature of −78° C. to 120° C. 4-Dimethylaminopyridine may aid the reaction.

### EXAMPLES

Below follows a number of non-limiting examples of compounds of the invention.

### General Methods

Starting materials used were available from commercial sources, or prepared according to literature procedures.

Microwave heating was performed in a Creator, Initiator or Smith Synthesizer Single-mode microwave cavity producing continuous irradiation at 2450 MHz.

¹H NMR spectra were recorded in the indicated deuterated solvent at either 300 MHz or 400 MHz. The 400 MHz spectra were obtained unless stated otherwise, using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR spectrometer equipped with a 4-nucleus probehead with Z-gradients. Chemical shifts are given in ppm down- and upfield from TMS. Resonance multiplicities are denoted s, d, t, q, m and br for singlet, doublet, triplet, quartet, multiplet, and broad respectively.

LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 2 mm ID×50 nm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm

Mass spectra (MS) were run using an automated system with atmospheric pressure chemical (APCI or CI) or electrospray (+ESI) ionization. Generally, only spectra where parent masses are observed are reported. The lowest mass major ion is reported for molecules where isotope splitting results in multiple mass spectral peaks (for example when chlorine is present).

GC-MS analyses were performed on a Agilent 6890N GC equipped with a Chrompack CP-Sil 5CB column (25 m×0.25 mm i.d. df=0.25)), coupled to an Agilent 5973 Mass Selective Detector operating in a chemical ionization (CI) mode and the MS was scanned between m/z 50-500. Alternatively, mass spectra were recorded using either a Hewlett Packard 5988A or a MicroMass Quattro-1 Mass Spectrometer and are reported as m/z for the parent molecular ion with its relative intensity.

HPLC assays were performed using an Agilent HP1100 Series system equipped with a Waters X-Terra MS, C₈ column (3.0×100 mm, 3.5 μm). The column temperature was set to 40° C. and the flow rate to 1.0 mL/min. The Diode Array Detector was scanned from 200-300 nm. A linear gradient was applied, run from 0% to 100% B in 4 min. Mobile phase A: 10 mM ammonium acetate in water/acetonitrile (95:5), mobile phase B: acetonitrile.

Preparative HPLC was performed on a Waters Auto purification HPLC-UV system with a diode array detector using a Waters XTerra MS C₈ column (19×300 mm, 7 μm) and a linear gradient of mobile phase B was applied. Mobile phase A: 0.1 M ammonium acetate in water/acetonitrile (95:5) and mobile phase B: acetonitrile. Flow rate: 20 mL/min.

Thin layer chromatography (TLC) was performed on Merch TLC-plates (Silica gel 60 F₂₅₄) and spots were UV visualized. Flash chromatography was performed using Merck Silica gel 60 (0.040-0.063 mm), or employing a Combi Flash® Companion™ system using RediSep™ normal-phase flash columns.

Compounds have been named using ACD/Name, version 8.08, software from Advanced Chemistry Development, Inc. (ACD/Labs), Toronto ON, Canada, www.acdlabs.com, 2004.

### Example 1

### tert-Butyl (2E)-3-(3-bromophenyl)but-2-enoate

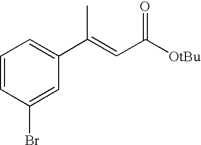

To a −78° C. stirred solution of tert-butyldimethylphosphonoacetate (21.9 mL, 0.111 mol) in tetrahydrofuran (150 mL) was added n-butyl lithium in hexanes (1.6 M, 72.0 mL, 0.116 mol) and the reaction was stirred at −78° C. for 10 min. To this mixture was added 3′-bromoacetophenone (13.4 mL, 0.100 mole) and the reaction allowed to warm to room temperature and was stirred for 18 h. The tetrahydrofuran was removed under reduced pressure to yield a solid. Hexanes (300 mL) added and the solids triturated for 1 h. The mixture was filtered through Celite and the filtrate concentrated under reduced pressure to give 28.9 g of the title compound. This was carried directly into the next reaction. ¹H NMR (300 MHz, DMSO-d₆): δ 7.71 (s, 1H); 7.53 (m, 2H); 7.36 (t, J=7.8 Hz, 1H); 6.05 (s, 1H); 2.44 (s, 3H); 1.47 (s, 9H).

### Example 2

### (2E)-3-(3-Bromophenyl)but-2-enoic acid

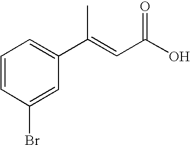

A solution of tert-butyl (2E)-3-(3-bromophenyl)but-2-enoate (28.9 g) in a mixture of trifluororacetic acid:dichloromethane (1:1, 300 mL) was stirred at room temperature for 15 min., and the solvents removed under reduced pressure. The resulting solid was triturated in hexanes (400 mL), filtered, and dried under vacuum to give 8.87 g (38% yield) of the title compound. ¹H NMR (300 MHz, DMSO-d₆): δ 7.72 (t, J=1.5 Hz, 1H); 7.53 (m, 2H); 7.37 (t, J=7.8 Hz, 1H); 6.11 (s, 1H); 2.46 (s, 3H).

### Example 3

### (2E)-3-(3-Bromophenyl)but-2-enoyl chloride

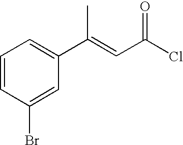

To a suspension of (2E)-3-(3-bromophenyl)but-2-enoic acid (1.00 g, 4.148 mmol) in 10 mL dichloromethane was added oxalyl chloride (434 uL, 4.98 mmol) followed by N,N-dimethylformamide (15 uL, 0.207 mmol) and the reaction was stirred at room temperature for 2 h. The solvent was removed under reduced pressure to give the title compound. ¹H NMR (300 MHz, DMSO-d₆): δ 7.63 (t, J=1.8 Hz, 1H); 7.57 (d, J=8.7 Hz, 1H); 7.43 (d, J=7.8 Hz, 1H); 7.29 (t, J=7.8 Hz, 1H); 6.44 (s, 1H); 2.51 (s, 3H).

### Example 4

### (2E)-3-(3-Bromophenyl)-N-cyano-N-methylbut-2-enamide

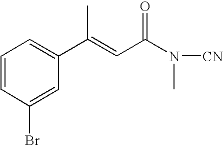

To a cooled (−60° C.) solution of cyanogen bromide (4.24 g, 40.00 mmol) in 100 mL tetrahydrofuran was added sodium carbonate (6.36 g, 60.00 mmol) followed by drop wise addition of a solution of methyl amine in tetrahydrofuran (2.0 M, 20.0 mL 40.00 mmol). The bath temperature was kept below −20° C. for 2 h. The reaction was filtered cold under a blanket of nitrogen through Celite and a solution of (2E)-3-(3-bromophenyl)but-2-enoyl chloride (5.19 g, 20.00 mmol) in 100 mL tetrahydrofuran was added to the filtrate. N,N-Diisopropylethylamine (4.2 mL, 24.00 mmol) was added and the reaction stirred at room temperature for 2 h. The solvent was removed under reduced pressure and the resulting oil put under high vacuum over night. The crude compound was purified using flash chromatography using dichloromethane as the eluent, to give 4.29 g (75% yield) of the title compound. ¹H NMR (300 MHz, DMSO-d₆): δ 7.76 (t, J=1.8 Hz, 1H); 7.65 (d, J=7.8 Hz, 1H); 7.58 (d, J=8.4 Hz, 1H); 7.42 (t, J=7.8 Hz, 1H); 6.65 (s, 1H); 3.22 (s, 3H); 2.44 (s, 3H).

### Example 5

### 6-(3-Bromophenyl)-2-imino-1-(4-methoxybenzyl)-3,6-dimethyltetrahydropyrimidin-4(1H)-one

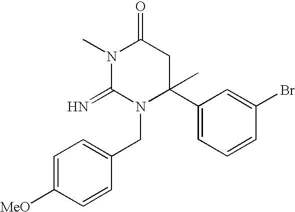

To a stirred solution of (2E)-3-(3-bromophenyl)-N-cyano-N-methylbut-2-enamide (12.77 g, 45.75 mmol) in 50 mL N,N-dimethylformamide was added 4-methoxybenzyl amine (14.9 mL, 114.4 mmol). After four hours the solvent was removed under reduced pressure and the resulting viscous oil put under high vacuum over night. The crude compound was purified using sequential flash chromatography. The first purification was using dichloromethane, methanol:dichloromethane (2.5:97.5) and methanol:dichloromethane (5:95) as the eluent to give 18.96 g crude product. The second purification was using diethyl ether, ethyl acetate, methanol:ethyl acetate (5:95) and methanol:ethyl acetate (10:90) as the eluent to give 15.48 g (81% yield) of the title compound. ¹H NMR (300 MHz, DMSO-d₆/TFA-d): δ 7.57 (m, 2H); 7.34 (m, 4H); 6.96 (d, J=8.7 Hz, 2H); 4.97 (dd, J=4.8 Hz, 2H); 3.78 (s, 3H); 3.58 (d, J=16.8 Hz, 1H); 3.30 (d, J=16.5 Hz, 1H); 3.20 (s, 3H); 1.65 (s, 3H); MS (APCI+) m/z 416.08 [M+1]⁺.

### Example 6

### 2-Amino-6-(3-bromophenyl)-3,6-dimethyl-5,6-dihydropyrimidin-4(3H)-one

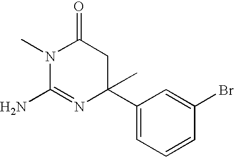

To a solution of 6-(3-bromophenyl)-2-imino-1-(4-methoxybenzyl)-3,6-dimethylteftahydropyrimidin-4(1H)-one (15.48 g, 37.18 mmol) in 150 mL acetonitrile was added 50 mL water followed by ammonium cerium nitrate (61.15 g, 111.55 mmol) and the reaction stirred for 18 h. Celite (32 g) was added followed by sodium bicarbonate (31.23 g, 371.8 mmol) and reaction stirred for 2 h. Additional Celite (15 g) was added after 1 h. The reaction was filtered through Celite and the filtrate concentrated under reduced pressure. The resulting orange solid was put under high vacuum. Purification by flash chromatography using methanol:dichloromethane:acetic acid (15:85:0.1) as the elunet. The resulting orange solid was triturated with methanol to give the first batch of the title compound. The solvents were removed from the filtrate under reduced pressure and the resulting orange solid was triturated with ethanol to give a second batch of the title compound. The batches were combined to give 8.75 g (79% yield) of the title compound. ¹H NMR (300 MHz, DMSO-d₆/TFA-d): δ 7.67 (s, 1H); 7.55 (m, 1H); 7.39 (m, 2H); 3.49 (d, J=16.2 Hz, 1H); 3.19 (d, J=16.5 Hz, 1H); 3.14 (s, 3H); 1.64 (s, 3H); MS (APCI+) m/z 296.0 [M+1]⁺.

### Example 7

### 3-Chloro-5-methoxyphenyl methanesulfonate

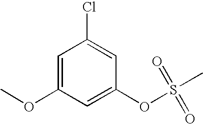

To a stirred solution of 3-chloro-5-methoxyphenol (500 mg, 3.15 mmol) in dichloromethane (20 mL) at 0° C., was added triethyl amine (485 μl, 3.47 mmol) followed by methanesulfonyl chloride (270 μl, 3.47 mmol). The reaction mixture was allowed to reach ambient temperature and was stirred for 18 h, before being washed with aqueous 1.2 M hydrochloric acid (20 mL). The organic phase was dried over magnesium sulphate, filtered and concentrated in vacuo to give 725 mg (97% yield) of the title compound, which was used without further purification. ¹H NMR (400 MHz, CDCl₃) δ 6.92-6.90 (m, 1H), 6.89-6.87 (m, 1H), 6.78-6.75 (m, 1H), 3.82 (s, 3H), 3.18 (s, 3H); MS (ESI) m/z 235 [M−1]⁻.

### Example 8

### 3-Methoxy-5-(4,4,5,5-tetramethyl-1,3,2-dioxaborolan-2-yl)phenyl methanesulfonate

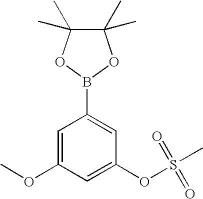

To a dried vial was added tris(dibenzylideneacetone)dipalladium(0) (13 mg, 0.013 mmol) and tricyclohexylphosphine (17 mg, 0.059 mmol) under an atmosphere of argon. Anhydrous dimethoxyethane (2.5 mL) was added and the resulting mixture was stirred for 30 min. 3-Chloro-5-methoxyphenyl methanesulfonate (100 mg, 0.42 mmol), 4,4,4′,4′,5,5,5′,5′-octamethyl-2,2′-bi-1,3,2-dioxaborolane (118 mg, 0.47 mmol) and potassium acetate (62 mg, 0.63 mmol) were added and the resulting mixture was irradiated in a microwave at 150° C. for 3 h. Upon cooling, water (5 mL) was added and the mixture was extracted with diethyl ether (3×4 mL). The combined organic extracts were concentrated in vacuo and purified by flash chromatography, using dichloromethane as the eluent, to give 110 mg (79% yield) of the title compound, which was used without further purification. ¹H NMR (400 MHz, CDCl₃) δ 7.30-7.24 (m, 2H), 6.96-7.93 (m, 1H), 3.84 (s, 3H), 3.14 (s, 3H), 1.34 (s, 12H); MS (ESI) m/z 329 [M+1]⁺.

### Example 9

### 3′-(2-Amino-1,4-dimethyl-6-oxo-1,4,5,6-tetrahydropyrimidin-4-yl)-5-methoxybiphenyl-3-yl methanesulfonate

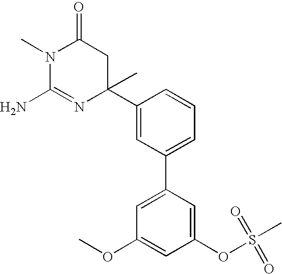

2-Amino-6-(3-bromophenyl)-3,6-dimethyl-5,6-dihydropyrimidin-4(3H)-one (90 mg, 0.30 mmol), 3-methoxy-5-(4,4,5,5-tetramethyl-1,3,2-dioxaborolan-2-yl)phenyl methanesulfonate (110 mg, 0.34 mmol), 1,1′-bis(diphenylphosphino)ferrocenedichloropalladium(II) (12.4 mg, 0.015 mmol) and cesium carbonate (297 mg, 0.91 mmol) were dissolved in a mixture of dimethoxyethane:water:ethanol (6:3:1, 3 mL) and irradiated in a microwave at 150° C. for 15 min. Upon cooling water was added and the mixture was extracted with diethyl ether (3×4 mL). The combined organic extracts were concentrated in vacuo, dissolved in acetonitrile and purified by preparative HPLC to give 7.6 mg (6% yield) of the title compound. ¹H NMR (400 MHz, CDCl₃) δ 7.66-7.61 (m, 1H), 7.54-7.49 (m, 1H), 7.48-7.42 (m, 1H), 7.38-7.32 (m, 1H), 7.15-7.12 (m, 1H), 7.12-7.08 (m, 1H), 6.87-6.83 (m, 1H), 3.88 (s, 3H), 3.37 (d, J=16.4 Hz, 1H), 3.29 (s, 3H), 3.22 (s, 3H), 3.03 (d, J=16.4 Hz, 1H), 1.74 (s, 3H); MS (ESI) m/z 418 [M+1]⁺.

### Example 10

### 8-(4,4,5,5-Tetramethyl-1,3,2-dioxaborolan-2-yl)chromane

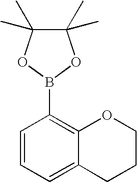

8-Bromochromane (described in Gerard H. Thomas et al. Tetrahedron. Lett. 1998, 39, 2219-2222, 426 mg, 2 mmol), 4,4,4′,4′,5,5,5′,5′-octamethyl-2,2′-bi-1,3,2-dioxaborolane (609 mg, 2.4 mmol), [1,1′-bis(diphenylphosphino)ferrocene]palladium(II) chloride dichloromethane adduct (50 mg, 0.06 mmol), potassium acetate (590 mg, 6 mmol) and 1,2-dimethoxyethan (3 mL) was irradiated in a microwave at 150° C. for 15 min. When cooled to ambient temperature the mixture was diluted with water (5 mL) and extracted with diethyl ether (3×20 mL). The crude product was purified by flash chromatography, using dichloromethane/methanol (95:5) as the eluent, to give 290 mg (56% yield) of the title compound. ¹H-NMR (400 MHz, DMSO-d₆): δ 7.32 (dd, J=7.3, 1.5 Hz, 1H), 7.12 (dd, J=7.4, 1.6 Hz, 1H), 6.76 (t, J=7.4 Hz, 1H), 4.12 (t, J=5.0 Hz, 2H), 2.71 (t, J=6.5 Hz, 2H), 1.92-1.84 (m, 2H), 1.25 (s, 12H).

### Example 11

### 2-Amino-6-[3-(3,4-dihydro-2H-chromen-8-yl)phenyl]-3,6-dimethyl-5,6-dihydropyrimidin-4(3H)-one hydrochloride

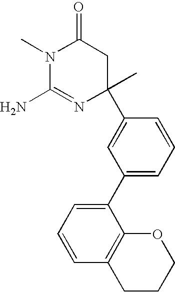

The title compound was synthesized as described for Example 9 starting from 2-Amino-6-(3-bromophenyl)-3,6-dimethyl-5,6-dihydropyrimidin-4(3B)-one and 8-(4,4,5,5-tetramethyl-1,3,2-dioxaborolan-2-yl)chromane). The crude product was purified by flash chromatography using acetonitrile/triethylamine (95:5) as the eluent. The hydrochloride salt was then prepared by dissolving the product in dichloromethane and treating the solution with hydrochloric acid (4.0 M in diethyl ether). The formed hydrochloride salt precipitated after addition of diethyl ether (15 mL) to yield 8 mg (8% yield) of the title compound. ¹H-NMR (400 MHz, DMSO-d₆): δ 10.29 (s, 1H), 8.57 (br s, 2H), 7.48 (s, 1H), 7.44-7.40 (m, 2H), 7.37-7.32 (m, 1H), 7.08 (d, J=7.5 Hz, 2H), 6.89 (t, J=7.5 Hz, 1H), 4.12 (t, J=4.8 Hz 2H), 3.48 (d, J=16.6 Hz, 1H), 3.18 (d, J=16.3 Hz, 1H), 3.09 (s, 3H), 2.81 (t, J=6.3 Hz, 2H), 1.98-1.89 (m, 2H), 1.63 (s, 3H); MS (ESI) m/z 350 [M+1]⁺.

### Assays

Compounds were tested in at least one of the following assays:

### β-Secretase Enzyme

The enzyme used in the IGEN Cleavage-, Fluorescent-, TR-FRET- and the BiaCore assay is described as follows:

The soluble part of the human β-Secretase (AA 1-AA 460) was cloned into the ASP2-Fc10-1-IRES-GFP-neoK mammalian expression vector. The gene was fused to the Fc domain of IgG1 (affinity tag) and stably cloned into HEK 293 cells. Purified sBACE-Fc is stored in Tris buffer, pH 9.2 and has a purity of 95%.

### IGEN Cleavage Assay

Enzyme is diluted 1:30 in 40 mM MES pH 5.0. Stock substrate is diluted to 12 μM in 40 mM MES pH 5.0. Compounds are diluted to the desired concentration in dimethylsulphoxide (final dimethylsulphoxide concentration in assay is 5%). The assay is done in a 96 well PCR plate from Greiner (#650201). Compound in dimethylsulphoxide (3 μL) is added to the plate, and then enzyme is added (27 μL) and pre-incubated with compound for 10 minutes. The reaction is started with substrate (30 μL). The final dilution of enzyme is 1:60 and the final concentration of substrate is 6 μM. After a 20 minute reaction at room temperature, the reaction is stopped by removing 10 μl of the reaction mix and diluting it 1:25 in 0.2 M Trizma-HCl, pH 8.0. Compounds are diluted and added to the plate by the Biomek FX or by hand, then all the rest of the liquid handling is done with on the Biomek 2000 instrument.

All antibodies and the streptavidin coated beads are diluted in PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/mL IGEN beads (Dynabeads M-280) and a 1:5000 dilution of ruthinylated goat anti-rabbit (Ru-GαR) antibody is added. The final dilution of neoepitope antibody is 1:20,000, the final dilution of Ru-GAR is 1:10,000 and the final concentration of beads is 0.1 mg/mL. The mixture is read on the IGEN instrument (BioVeris) with the Abbiochemial assay program after a 2-hour incubation with shaking at room temperature.

### Fluorescent Assay

Enzyme is diluted 1:25 in 40 mM MS pH 5.0. Stock substrate (Dabcyl) is diluted to 30 μM in 40 mM MES pH 5.0. Enzyme and substrate stock solutions are kept on ice until placed in the stock plates. The Biomek FX instrument is used to do all liquid handling. Enzyme (9 μL) together with 1 μL of compound in dimethylsulphoxide is added to the plate and pre-incubated for 10 minutes. When a dose response curve is being tested for a compound, the dilutions are done in neat dimethylsulphoxide. Substrate (10 μL) is added and the reaction proceeds in the dark for 25 minutes at room temperature. The assay is done in a Corning 384 well round bottom, low volume, non-binding surface (Corning #3676). The final dilution of enzyme is 1:50, and the final concentration of substrate is 150 μM (Km of 25 μM). The fluorescence of the product is measured on a Victor II plate reader with an excitation wavelength of 360 nm and an emission wavelength of 485 nm using the protocol for labelled Edans peptide. The dimethylsulphoxide control defines 100% activity level and 0% activity is defined by exclusion of the enzyme (using 40 mM MES pH 5.0 buffer instead).

### TR-FRET Assay

Dilute the enzyme (truncated form) to 6 μg/mL (stock 1.3 mg/mL) and the substrate (Europium)CEVNLDAEFK (Qsy7) to 200 nM (stock 60 μM) in reaction buffer (NaAcetate, chaps, triton x-100, EDTA pH4.5). The Biomek FX is used for all liquid handling and the enzyme and substrate solutions are kept on ice until they are placed in Biomek FX. Enzyme (9 μl) is added to the plate then 11 of compound in dimethylsulphoxide is added, mixed and pre-incubated for 10 minutes. Substrate (10 μl) is then added, mixed and the reaction proceeds in the dark for 15 minutes at room temperature. The reaction is stopped with the addition of Stop solution (7 μl, NaAcetate pH 9). The fluorescence of the product is measured on a Victor II plate reader with an excitation wavelength of 340 nm and an emission wavelength of 615 nm. The assay is done in a Costar 384 well round bottom, low volume, non-binding surface (Corning #3676). The final concentration of the enzyme is 0.3 nM; the final concentration of substrate is 100 nM (Km of ˜250 nM). The dimethylsulphoxide control defines the 100% activity level and 0% activity is defined by only addition of the peptide substrate. A control inhibitor is also used in dose response assays and has an IC50 of 575 nM.

### Beta-Secretase Whole Cell Assay

### Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/mL of zeocin. Limited dilution cloning was performed to generate homogeneous cell lines. Clones were characterized by levels of APP expression and Aβ secreted in the conditioned media using an ELISA assay developed in-house.

### Cell Culture

HEK293 cells stably expressing human wild-type APP (HEK293-APP695) were grown at 37° C. in DMEM containing 4500 g/L glucose, GlutaMAX and sodium pyruvate supplemented with 10% FBS, 1% non-essential amino acids and 0.1 mg/mL of the selection antibiotic zeocin.

### Aβ40 Release Assay

Cells were harvested at 80-90% confluence and seeded at a concentration of 0.2×10⁶ cells/mL, 100 mL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) and containing test compounds in a final dimethylsulphoxide concentration of 1%. Cells were exposure to test compounds for 24 h at 37° C., 5% CO2. To quantify the amount of released Aβ, 100 μL cell medium was transferred to a round bottom polypropylene 96-well plate (assay plate). The cell plate was saved for ATP assay as described in ATP assay below. To the assay plate, 50 μL of primary detection solution containing 0.5 μg/mL of the rabbit anti-Aβ40 antibody and 0.5 μg/mL of the biotinylated monoclonal mouse 6E10 antibody in DPBS with 0.5% BSA and 0.5% Tween-20 was added per well and incubated over night at 4° C. Then, 50 μL of secondary detection solution containing 0.5 μg/mL of a ruthenylated goat anti-rabbit antibody and 0.2 mg/mL of streptavidin coated Dynabeads was added per well. The plate was vigorously shaken at room temperature for 1-2 h. The plate was then measured for electro-chemiluminescence counts in an IGEN M8 Analyzer. An Aβ standard curve was obtained using standards at concentrations 20, 10, 2 and 0.2 ng Aβ/mL in the cell culture medium with penicillin and streptomycin (100 U/mL, 100 μg/mL, respectively).

### ATP Assay

As indicated above, after transferring 100 μL medium from the cell plate for Aβ40 detection, the plate was used to analyse cytotoxicity using the ViaLight™ Plus cell proliferation/cytotoxicity kit from Cambrex BioScience that measures total cellular ATP. The assay was performed according to the manufacture's protocol. Briefly, 50 μL cell lysis reagent was added per well. The plates were incubated at room temperature for 10 min. Two min after addition of 100 μL reconstituted ViaLight™ Plus ATP reagent, the luminescence was measured in a Wallac Victor² 1420 multilabel counter.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/mL in 20 mM Na Acetate pH 4.5, and then the solutions were centrifuged at 14K rpm to remove any particulates. Carboxyl groups on the dextran layer were activated by injecting a one to one mixture of 0.5M N-ethyl-N′(3-dimethylaminopropyl)-carbodiimide (EDC) and 0.5M N-hydroxysuccinimide (NHS) at 5 uL/minute for 7 minutes. Then the stock solution of the control peptide was injected in channel 1 for 7 minutes at 5 uL/min., and then the remaining activated carboxyl groups were blocked by injecting IM ethanolamine for 7 minutes at 5 uL/minute.

### Assay Protocol

The BACE Biacore assay was done by diluting BACE to 0.5 μM in Na Acetate buffer at pH 4.5 (running buffer minus dimethylsulplioxide). The diluted BACE was mixed with dimethylsulphoxide or compound diluted in dimethylsulphoxide at a final concentration of 5% dimethylsulphoxide. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethylsulphoxide control was defined as 100% and the effect of the compound was reported as percent inhibition of the dimethylsulphoxide control.

hERG Assay

### Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The is reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and BEPES 5 (all Sigma-Aldrich; pH 7.25-7.30 using 10 M KOH). After priming and de-bubbling, the electronics-head A-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound BERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV; see FIG. 1c) and then making the necessary offset adjustment in the IonWorks™ HT software. The current signal was sampled at 2.5 kHz.

Pre- and post-scan hERG-current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan hERG tail current amplitude >150 pA; post-scan seal resistance >60 MΩ. The degree of inhibition of the hERG current was assessed by dividing the post-scan hERG current by the respective pre-scan hERG current for each well.

### Results

Typical Kᵢ values for the compounds of the present invention are in the range of about 1 to about 100,000 nM. Biological data on an example is given below in Table 1.

TABLE 1

| Example No. | IC50 in TR-FRET assay |
|---|---|
| 9 | 703 nM |

1. A compound of formula I: 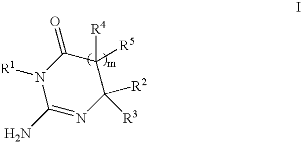
wherein
R¹ is selected from hydrogen, C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₁₆alkylC₃₋₆cycloalkyl, C₁₋₁₆alkylaryl, C₁₋₆alkylheteroaryl and C₁₋₆alkylheterocyclyl, wherein the C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl is optionally substituted with one, two or three A;
R² is selected from hydrogen, nitro, cyano, -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, and -Q-C₁₋₆alkylheterocyclyl, wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, and -Q-C₁₋₆alkylheterocyclyl is optionally substituted by one, two or three R⁷;
-Q- is a direct bond, —CONH—, —CO—, —CON(C₁₋₆alkyl)-, —CON(C₃₋₆cycloalkyl)-, —SO—, —SO₂—, —SO₂NH—, —SO₂N(C₁₋₆alkyl)-, —SO₂N(C₃₋₆cycloalkyl)-, —NHSO₂—, —N(C₁₋₆alkyl)SO₂—, —NHCO—, —N(C₁₋₆alkyl)CO—, —N(C₃₋₆cycloalkyl)CO— or —N(C₃₋₆cycloalkyl)SO₂—;
R³ is (C(R²⁷)(R²⁸))ₙR⁶, C₂₋₄alkenylR⁶, C₂₋₄alkynylR⁶, C₅₋₇cycloalkenylR⁶, nitro or cyano and if n>1 then each C(R²⁷)(R²⁸) is independent of the others;
R²⁷ and R²⁸ are independently selected from hydrogen, C₁₋₆alkyl, cyano, halo and nitro; or R²⁷ and R²⁸ together form oxo, C₃₋₆cycloalkyl or heterocyclyl;
R⁴ and R⁵ are selected from hydrogen, nitro, cyano, -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, and -Q-C₁₋₆alkylheterocyclyl, wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, and -Q-C₁₋₆alkylheterocyclyl is optionally substituted by one, two or three R⁷; or
R⁴ and R⁵ may optionally join together to form a C₃₋₇cycloalkyl, C₅₋₇cycloalkenyl or heterocycle ring optionally substituted by one, two or three R⁷; or
R⁴ or R⁵, which are connected to the carbon directly adjacent to the carbon to which R² and R³ are connected, join together with either R² or R³ to form a C₃₋₇cycloalkyl, C₅₋₇cycloalkenyl or heterocycle ring optionally substituted by one, two or three R⁷;
R⁶ is selected from methyl, C₃₋₆cycloalkyl, heterocyclyl, aryl and heteroaryl wherein each of the said methyl, C₃₋₆cycloalkyl, heterocyclyl, aryl of and heteroaryl is optionally substituted with from one to four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with from one to four A with the proviso that the bicyclic ring is not an indane, benzo[1,3]dioxole or 2,3-dihydrobenzo[1,4]-dioxine ring system;
R⁷ is selected from halogen, nitro, CHO, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR³R⁹, OC₂₋₆alkylOC₂₋₆alkylNR³R⁹, NR³OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR³(CO)R⁹, C₀₋₆alkylNR³ (CO)R⁹, O(CO)NR⁸R⁹, NR⁸(CO)OR⁹, NR⁸(CO)NR⁸R⁹, O(CO)OR⁸, O(CO)R⁸, C₀₋₆alkylCOR³, OC₁₋₆alkylCOR³, NR⁸(CO)(CO)R⁸, NR⁸(CO)(CO)NR⁸R⁹, C₀₋₆alkylSR⁸, C₀₋₆alkyl(SO₂)NR³R⁹, OC₁₋₆alkylNR³(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, SO₃R⁸, C₀₋₆alkylNR⁸(SO₂)NR⁸R⁹, C₀₋₆alkylNR³(SO)R⁹, OC₂₋₆alkylNR⁸(SO)R⁸, OC₁₋₆alkylSO₂R³, C₁₋₆alkylSO₂R³, C₀₋₆alkylSOR³, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl, wherein any C₁₋₁₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with from one to four A with the proviso that said bicyclic ring system is not an indane, benzo[1,3]dioxole or 2,3-dihydrobenzo[1,4]-dioxine ring system;
R¹⁴ is selected from halogen, nitro, CHO, C₀₋₆alkylCN, OC₁₋₆alkylCN, C₀₋₆alkylOR³, OC₁₋₁₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR³R⁹, OC₂₋₆alkylOC₂₋₆alkylNR³R⁹, NR³OR⁹, C₀₋₆alkylCO₂R³, OC₁₋₆alkylCO₂R³, C₀₋₆alkylCONR³R⁹, OC₁₋₆alkylCONR³R⁹, OC₂₋₆alkylNR³(CO)R⁹, C₀₋₆alkylNR³ (CO)R⁹, O(CO)NR⁸R⁹, NR⁸(CO)OR⁹, NR⁸(CO)NR⁸R⁹, O(CO)OR⁸, O(CO)R⁸, C₀₋₆alkylCOR³, OC₁₋₆alkylCOR³, NR⁸(CO)(CO)R⁸, NR⁸(CO)(CO)NR⁸R⁹, C₀₋₆alkylSR⁸, C₀₋₆alkyl(SO₂)NR⁸R⁹, OC₂₋₆alkylNR⁸(SO₂)R⁹, OC₀₋₆alkyl(SO₂)NR³R⁹, C₀₋₆alkyl(SO)NR³R⁹, OC₁₋₆alkyl(SO)NR³R⁹, OSO₂R⁸, OR⁸, SO₃R⁸, C₀₋₆alkylNR⁸(SO₂)NR⁸R⁹, C₀₋₆alkylNR³(SO)R⁹, OC₂₋₆alkylNR⁸(SO)R⁸, OC₁₋₆alkylSO₂R³, C₁₋₆alkylSO₂R³, C₀₋₆alkylSOR³, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl may be optionally substituted by from one to four A;
R⁸ and R⁹ are independently selected from hydrogen, C₁₋₆alkyl, C₂₋₆alkenyl, C₂₋₆alkynyl, fluoromethyl, difluoromethyl, trifluoromethyl, fluoromethoxy, difluoromethoxy, trifluoromethoxy, C₀₋₆alkylC₃₋₆cycloalkyl, C₀₋₆alkylaryl, C₀₋₆alkylheteroaryl, C₀₋₆alkylheterocyclyl and C₁₋₆alkylNR¹⁰R¹¹, wherein the C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl and C₀₋₆alkylheterocyclyl are optionally substituted by A; or
R⁸ and R⁹ may together form a 4 to 6 membered heterocyclic ring containing one or more heteroatoms selected from N, O and S that is optionally substituted by A; whenever two R⁸ groups occur in the structure then they may optionally together form a 5 or 6 membered heterocyclic ring containing one or more heteroatoms selected from N, O and S, that is optionally substituted by A;
R¹⁰ and R¹¹ are independently selected from hydrogen, C₁₋₆alkyl, C₃₋₆alkenyl, C₃₋₆alkynyl, C₀₋₆alkylC₃₋₆cycloalkyl, C₀₋₆alkylaryl, C₀₋₆alkylheterocyclyl and C₀₋₆alkylheteroaryl, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, and C₀₋₆alkylheterocyclyl are optionally substituted by A; or
R¹⁰ and R¹¹ may together form a 4 to 6 membered heterocyclic ring containing one or more heteroatoms selected from N, O and S optionally substituted by A;
m is 1 or 2;
n is 0, 1, 2 or 3;
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, OC₂₋₆alkylNR¹²R¹³, NR¹²R¹³, CONR¹²R¹³, NR¹²(CO)R¹³, O(CO)C₁₋₆alkyl, (CO)OC₁₋₆alkyl, COR¹², (SO₂)NR¹²R¹³, NSO₂R¹², SO₂R¹², SOR¹², (CO)C₁₋₆alkylNR¹²R¹³, (SO₂)C₁₋₆alkylNR¹²R¹³, OSO₂R¹², and SO₃R¹² wherein the C₁₋₆alkyl, C₂₋₆alkenyl, C₂₋₆alkynyl, C₀₋₆alkylaryl, C₀₋₆alkylheteroaryl, C₀₋₆alkylheterocyclyl and C₀₋₆alkylC₃₋₆cycloalkyl groups may be optionally substituted with halo, OSO₂R¹², SO₃R¹², nitro, cyano, OR¹², C₁₋₆alkyl, fluoromethyl, difluoromethyl, trifluoromethyl, fluoromethoxy, difluoromethoxy or trifluoromethoxy;
R¹² and R¹³ are independently selected from hydrogen, C₁₋₆alkyl, C₃₋₆cycloalkyl, aryl, heteroaryl and heterocyclyl wherein said C₁₋₆alkyl, C₃₋₆cycloalkyl, aryl, heteroaryl and heterocyclyl is optionally substituted by one, two or three hydroxy, cyano, halo or C₁₋₃alkyloxy; or
R¹² and R¹³ may together form a 4 to 6 membered heterocyclic ring containing one or more heteroatoms selected from N, O and S optionally substituted by hydroxy, C₁₋₃alkyloxy, cyano or halo;
provided that either any of the aryl or heteroaryl groups in R¹, R², R³, R⁴ or R⁵ is substituted with a OSO₂R⁸, SO₃R⁸, OSO₂R¹² or SO₃R¹² group; or
provided that when any of the individual aryl or heteroaryl groups in R¹, R², R³, R⁴ or R⁵ are fused with a 4, 5, 6 or 7 membered cycloalkyl, cycloalkenyl or heterocyclyl group to form a bicyclic ring system where the bicyclic ring system is optionally substituted with between from one and to four A, the bicyclic ring is not an indane, benzo[1,3]dioxole or 2,3-dihydrobenzo[1,4]-dioxine ring system; or
provided that when R¹ is C₃₋₆alkynyl or C₅₋₇cycloalkenyl, said groups are optionally substituted with one, two or three A; or
provided that Q is selected from —NHSO₂—, —N(C₁₋₆alkyl)SO₂—, —SO₂NH—, —SO₂N(C₁₋₆alkyl)-, —SO₂N(C₃₋₆cycloalkyl)- and —N(C₃₋₆cycloalkyl)SO₂—; or
provided that R³ is selected from C₂₋₄alkenylR⁶, C₂₋₄alkynylR⁶, C₅₋₇cycloalkenylR⁶ and nitro; or
provided that R² is selected from nitro, C₂₋₆alkynyl, C₅₋₇cycloalkenyl and C₂₋₆alkenyl group where the C₂₋₆alkynyl, C₅₋₇cycloalkenyl and C₂₋₆alkenyl group is optionally substituted by one, two or three R⁷; or
provided that R⁴ or R⁵ are independently selected from nitro, C₂₋₆alkynyl, C₅₋₇cycloalkenyl of and C₂₋₆alkenyl group where the C₂₋₆alkynyl, C₅₋₇cycloalkenyl and C₂₋₆alkenyl group is optionally substituted by one, two or three R⁷; or
provided that when Q is —SO— or —SO₂— that the said —SO— or —SO₂— group connect to carbons;
as a free base or a pharmaceutically acceptable salt, solvate or solvate of a salt thereof.

2. A compound according to claim 1, wherein R¹ is C₁₋₆alkyl.

3. A compound according to claim 2, wherein C₁₋₆alkyl is methyl.

4. A compound according to claim 1, wherein -Q- in R² represents a direct bond.

5. A compound according to claim 4, wherein R² is C₁₋₆alkyl.

6. A compound according to claim 5, wherein C₁₋₆alkyl is methyl.

7. A compound according to claim 1, wherein R³ is (C(R²⁷)(R²⁸))ₙR⁶.

8. A compound according to claim 7, wherein n is 0.

9. A compound according to claim 7, wherein R⁶ (of R³) is aryl, substituted with one R⁷.

10. A compound according to claim 9, wherein R⁷ is C₀₋₆alkylaryl, wherein C₀₋₆ alkylaryl is substituted by one or more R¹⁴, or wherein any of the individual aryl groups is fused with a 6 membered heterocyclyl group to form a bicyclic ring system.

11. A compound according to claim 10, wherein said C₀₋₆alkylaryl is phenyl.

12. A compound according to claim 11, wherein R¹⁴ is independently selected from OSO₂R⁸ and OR⁸.

13. A compound according to claim 12, wherein R⁸ is C₁₋₆alkyl.

14. A compound according to claim 11, wherein said phenyl is fused with a 6 membered heterocyclyl group to form a bicyclic ring system.

15. A compound according to claim 1, wherein R⁴ is hydrogen.

16. A compound according to claim 1, wherein m is 1.

17. A compound according to claim 1, wherein R¹ is C₁₋₆alkyl, -Q- in R² represents a direct bond and R² is C₁₋₆alkyl, R³ is (C(R²⁷)(R²⁸))ₙR⁶, n is 0, R⁶ (of R³) is aryl, substituted with one R⁷, R⁷ is phenyl substituted by one or more R¹⁴, R¹⁴ is independently selected from OSO₂R⁸ and OR⁸, R⁸ is C₁₋₆alkyl, R⁴ is hydrogen and m is 1.

18. A compound according to claim 1, wherein R¹ is C₁₋₆alkyl, -Q- in R² represents a direct bond and R² is C₁₋₆alkyl, R³ is (C(R²⁷)(R²⁸))ₙR⁶, n is 0, R⁶ (of R³) is aryl, substituted with one R⁷, R⁷ is phenyl fused with a 6 membered heterocyclyl group to form a bicyclic ring system, R⁴ is hydrogen and m is 1.

19. A compound being:
3′-(2-Amino-1,4-dimethyl-6-oxo-1,4,5,6-tetrahydropyrimidin-4-yl)-5-methoxybiphenyl-3-yl methanesulfonate; or
2-Amino-6-[3-(3,4-dihydro-2H-chromen-8-yl)phenyl]-3,6-dimethyl-5,6-dihydropyrimidin-4(3H)-one hydrochloride.

20. A pharmaceutical composition comprising a compound according to claim 1 and a pharmaceutically acceptable excipient, carrier or diluent.

21-25. (canceled)

26. A method of inhibiting activity of BACE comprising contacting said BACE with a compound of claim 1.

27. A method of treating an Aβ-related pathology in a mammal, comprising administering to said mammal a therapeutically effective amount of a compound of claim 1.

28. The method of claim 27, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, an attention deficit symptom associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

29. The method of claim 27, wherein said mammal is a human.

30. A method of treating an Aβ-related pathology in a mammal, comprising administering to said mammal a therapeutically effective amount of a compound of claim 1 and at least one cognitive enhancing agent, memory enhancing agent, or choline esterase inhibitor.

31. The method of claim 30, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, an attention deficit symptom associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

32. The method of claim 30, wherein said mammal is a human.
